(19) United States
(12) Patent Application Publication
(10) Pub. No.: US 2024/0038339 A1
(43) Pub. Date: Feb. 1, 2024

(54) BAYESIAN SEX CALLER

(71) Applicant: Myriad Women's Health, Inc., South San Francisco, CA (US)

(72) Inventors: Albert Lee, South San Francisco, CA (US); Kevin Haas, South San Francisco, CA (US); Kevin D'Auria, South San Francisco, CA (US)

(73) Assignee: Myriad Women's Health, Inc., South San Francisco, CA (US)

(21) Appl. No.: 18/020,416

(22) PCT Filed: Aug. 5, 2021
(86) PCT No.: PCT/US2021/044644

§ 371 Date: Feb. 8, 2023

Related U.S. Application Data
(60) Provisional application No. 63/151,451, filed on Feb. 19, 2021.
(60) Provisional application No. 63/063,401, filed on Aug. 9, 2020.

(51) Int. Cl. G16B 40/20; G16B 20/10; C12Q 1/6827; C12Q 1/6879

(52) U.S. Cl. CPC G16B 40/20; G16B 20/10; C12Q 1/6827; C12Q 1/6879

(57) ABSTRACT

A method and system for analyzing sex-chromosome aneuploidies of an individual are provided. In one embodiment, a method comprises training a neural network model based on predetermined information related to at least one sex chromosome. The method also comprises determining a respective sex-chromosome status based on a normalized read depth for a gene in a genome of the individual using a machine learning algorithm. The machine learning algorithm is configured to receive, as inputs, the normalized read depth, and output the respective sex-chromosome status of the individual. In another embodiment, a system I is provided including a neural network model trained based on predetermined information related to at least one sex chromosome and is adapted to determine a respective sex-chromosome status based on a normalized read depth for a gene in a genome of the individual using a machine learning algorithm.

### CROSS-REFERENCE TO RELATED APPLICATIONS

This application claims the benefit of U.S. provisional application No. 63/063,401, filed 9 Aug. 2020, and U.S. provisional application No. 63/151,451 filed 19 Feb. 2021, each application of which is hereby incorporated by reference as though fully set forth herein.

### BACKGROUND

### a. Field

The disclosure relates generally to improved sex chromosome analysis, such as for noninvasive prenatal screening.

### b. Background

Circulating throughout the bloodstream of a pregnant woman and separate from cellular tissue are small pieces of DNA, often referred to as cell-free DNA (cfDNA). The cfDNA in the maternal bloodstream includes cfDNA from both the mother (i.e., maternal cfDNA) and the fetus (i.e., fetal cfDNA). The fetal cfDNA originates from the placental cells undergoing apoptosis and constitutes up to 25% of the total circulating cfDNA, with the balance originating from the maternal genome.

Recent technological developments have allowed for noninvasive prenatal screening of chromosomal aneuploidy in the fetus by exploiting the presence of fetal cfDNA circulating in the maternal bloodstream. Noninvasive methods relying on cfDNA sampled from the pregnant woman's blood serum are particularly advantageous over chorionic villi sampling or amniocentesis, both of which risk substantial injury and possible pregnancy loss.

Determination of the fraction of fetal cfDNA taken from a maternal test sample allows for screening of fetal aneuploidy. The fetal fraction for male pregnancies (i.e., a male fetus) can be determined by comparing the amount of Y chromosome from the cfDNA, which can be presumed to originate from the fetus, to the amount of one or more genomic regions that are present in both maternal and fetal cfDNA. Determination of the fetal fraction for female pregnancies (i.e., a female fetus) is more complex, as both the fetus and the pregnant mother have similar sex-chromosome dosage and there are few features to distinguish between maternal and fetal DNA. Methylation differences between the fetal and maternal DNA can be used to estimate the fetal fraction of cfDNA. See, for example, Chim et al., PNAS USA, 102:14753-58 (2005). In another method, the fraction of fetal cfDNA can be determined by sequencing polymorphic loci to search for allelic differences between the maternal and fetal cfDNA. See, for example, U.S. Pat. No. 8,700,338. However, as explained in U.S. Pat. No. 8,700,338 (col. 18, lines 28-36), use of polymorphic loci to determine fetal fraction can become unreliable when the fetal fraction drops below 3%. See also Ryan et al., Fetal Diag. & Ther., vol. 40, pp. 219-223 (Mar. 31, 2016), which describes setting a threshold for “no call” when the fetal fraction is below 2.8%. United States Patent Publication no. 2018/0089364 entitled “Noninvasive Prenatal Screening Using Dynamic Iterative Depth Optimization.”

The disclosures of all publications referred to herein are each hereby incorporated herein by reference in their entireties. To the extent that any reference incorporated by references conflicts with the instant disclosure, the instant disclosure shall control.

Sex-chromosome aneuploidies (SCA) analysis in a Prenatal Screen serves two purposes: 1) predicting the sex of a fetus (“sex calling”) and 2) screening for sex-chromosome (chromosomes X and/or Y) aneuploidies. We have updated the underlying sex-calling algorithm in order to 1) predicting the sex of each fetus individually in a twin pregnancy (“twin sex calling”) and 2) incorporate two additional variables to identify complex cases, including those likely involving a vanishing twin and maternal mosaicism. These improvements provide a model that is easy to extend and more robust, due to the principled Bayesian theory to provide improved performance and accuracy, while maintaining current production performance.

### BRIEF SUMMARY

Systems and methods for analyzing sex-chromosomes are provided. In various implementations, for example, sex-chromosome aneuploidies (SCA) analysis in a prenatal screen is provided to perform at least one of the following: 1) sex calling, 2) screening for sex-chromosome (chromosomes X and/or Y) aneuploidies, 3) perform twin sex calling, and 4) incorporate two or more additional variables to identify complex cases, including those that may involve a vanishing twin and maternal mosaicism. The systems and methods utilize a Bayesian network trained on information related to at least one sex chromosome and trained and calibrated on a cohort of historical samples to establish statistical parameters and thresholds of confidence.

Fetal maternal samples taken from pregnant women include both maternal cell-free DNA and fetal cell-free DNA. Described herein are methods for determining a chromosomal abnormality of a test chromosome or a portion thereof in a fetus by analyzing a test maternal sample of a woman carrying said fetus, wherein the test maternal sample comprises fetal cell-free DNA and maternal cell-free DNA. The chromosomal abnormality can be, for example, aneuploidy or the presence of a microdeletion. In some embodiments, the chromosomal abnormality is determined by measuring a dosage of the test chromosome or portion thereof in the test maternal sample, measuring a fetal fraction of cell-free DNA in the test maternal sample, and determining an initial value of likelihood that the test chromosome or the portion thereof in the fetal cell-free DNA is abnormal based on the measured dosage, an expected dosage of the test chromosome or portion thereof, and the measured fetal fraction.

In one implementation, for example, a system and method adapted to analyze sex-chromosome aneuploidies of an individual is provided. The aneuploidies may include the following types by example: XXY, XYY, X, or XXX (referring to the number of X and Y chromosomes in the fetus) that are copies of chromosomes which are abnormal from the typical female XY and male XX chromosomes. In this implementation, a Bayesian network is adapted to be trained based on predetermined information related to at least one sex chromosome. A machine learning module is used to determine a sex-chromosome status based on a normalized read depth of the individual for the gene. The machine learning module is configured to receive inputs, such as the normalized read depth per chromosome, fetal fraction, and total number of sequencing reads and output the respective sex-chromosome status of the individual.

The foregoing and other aspects, features, details, utilities, and advantages of the present invention will be apparent from reading the following description and claims, and from reviewing the accompanying drawings.

### DETAILED DESCRIPTION

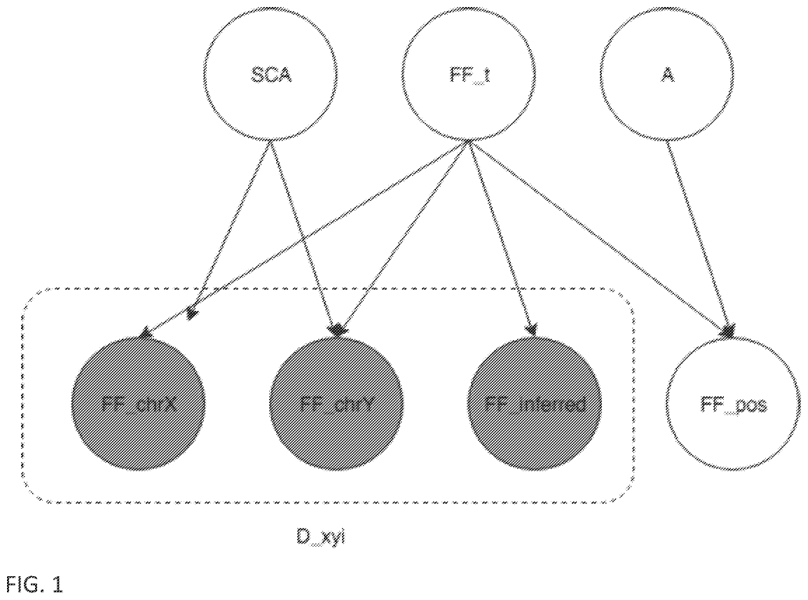

FIG. 1 is a block diagram showing an example graphical model for observed and unobserved variables for a Bayesian network adapted to analyze sex chromosomes. In this implementation, the graphical model includes a plurality of observed variables in a bottom row and a plurality of unobserved variables in a top row. In this example, there are four observed variables including depth and three probabilities as shown in Table 1 that can be calculated given the “depth-scaling parameters” fit from historical data.

TABLE 1

| Variable | Probability |
|---|---|
| FF_{chrX} | P(FF_{chrX}\|FFₜ, SCA) |
| FF_{chrY} | P(FF_{chrY}\|FFₜ, SCA) |
| FF_{inferred} | P(FF_{inferred}\|FFₜ) |

The variables in Table 1 include the fetal fraction as provided from normalized map reads on chrX versus chrY versus a whole genome inference.

In Table 1, FFₜ is the true unobserved fetal fraction, FF_{chrX} and FF_{chrY} is the deviation from expected normalized read depth for chromosome X and Y respectively, and SCA is a sex call. After selecting priors, the priors P(FFₜ), and P (SCA), other useful probabilities can also be derived. In one example, it can be assumed that all four parameters have Gaussian error with means and variances. FFₜ can be assumed to follow beta distribution, and its parameters fit using a maximum likelihood model on previously observed data with known fetal fraction. Elements in the sample space are the following:
SCA: the sex chromosome aneuploidy (aka sex chromosome analysis) is one of XX, XY, XXX, X, XXY, or XYY
FFₜ, the true fraction is bounded between 0.0 and 1.0
FF_{chrX}, FF_{chrY}, and FFₚₒₛ are theoretically unbounded reals, but practically will be between −1.0 and 1.0
FF_{inferred}, the inferred fetal fraction has a lower bound of 0.0 because the algorithm to produce it clamps all predictions at 0..0. it is theoretically unbounded on the high end, but practically, it not go above 1.0 unless there is a problem with the sample.

The relationships between the observed variables in Table 1 and the unobserved variables (SCA, and FFₜ) are shown in the graphical model of FIG. 1. A posterior probability of sex calls is the following:

$$\begin{matrix}P ( {D}_{xyi} \vert {FF}_{t} , {SCA}_{j} ) = P ( {FF}_{chrX} \vert {FF}_{t} , {SCA}_{j} ) P ( {FF}_{chrY} \vert {FF}_{i} , \\ {SCA}_{j} ) P ( {FF}_{inferred} \vert {FF}_{t} ) \\ P ( {D}_{xyi} \vert {SCA}_{j} ) = {\int }_{0}^{1} P ( {D}_{xyi} \vert {FF}_{i} = x , {SCA}_{j} ) P ( {FF}_{i} = x ) dx \\ P ( {D}_{xyi} ) = {\sum }_{i ∈ SCA} P ( {D}_{xyi} \vert {SCA}_{j} ) P ( {SCA}_{j} ) \\ P ( {SCA}_{j} \vert {D}_{xyi} ) = p ( {D}_{xyi} \vert {SCA}_{j} ) P ( {SCA}_{j} ) / P ( {D}_{xyi} )\end{matrix} \\ where {D}_{xyi} is the set of data for {FF}_{chrX} , {FF}_{chrY} , and {FF}_{inferred} .$$

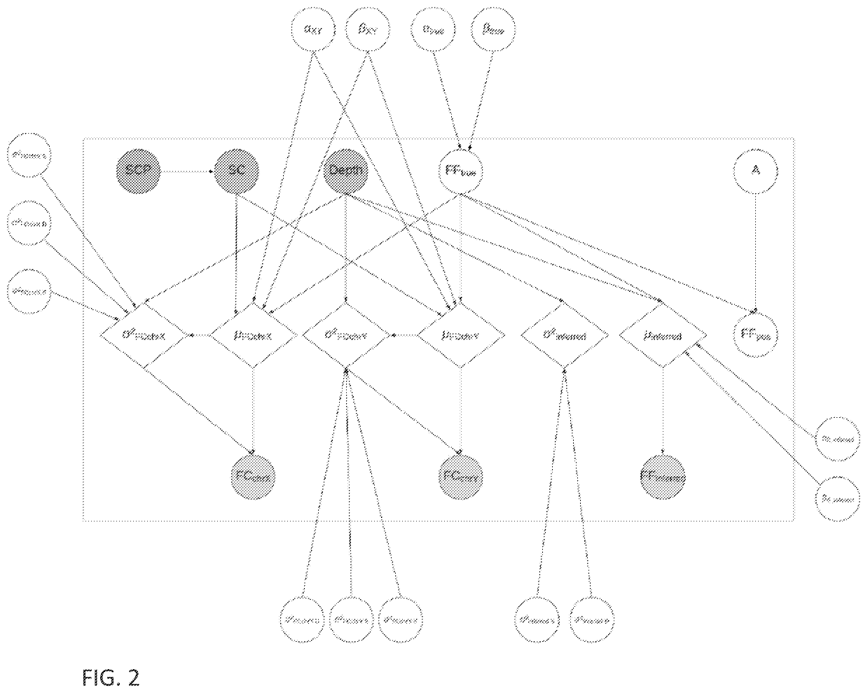

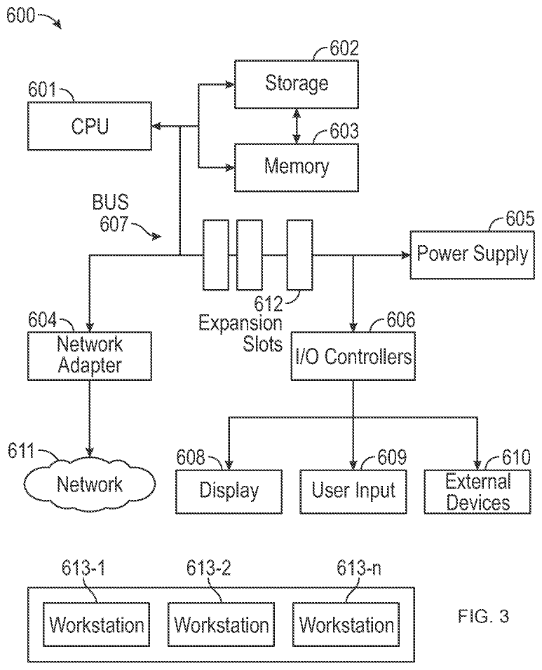

FIG. 2 is a block diagram showing an example plate notation for a Bayesian network adapted to analyze sex chromosomes. In this implementation, the Bayesian network includes a plurality of interconnected nodes shown in the plate notation that represent variables of the Bayesian network. Given the following information for a sample, Fold Change Chromosome X, Fold Change Chromosome Y, fetal fraction inferred (FF_{inferred}), and depth, probabilities for sex chromosomes, such as XX, XXXX, XY, XXY, and XYY can be determined. A sex call can be made based on the call with the highest probability. Alternatively, where no call has a probability above a predetermined threshold (e.g., 50%), a “No Call” may be made and the determination flagged for further review (e.g., human or other system review).

In the Bayesian network shown in FIG. 1, the model includes the following specification:

p_{sex call}˜Dirichlet(w) where, w=(w₁, . . . , wₖ), k=6

sex call˜Categorical(p_{sex call})

FFₜ˜Beta(α_{FF}, β_{FF})

FF_{inferred}˜ [symbol] (μ_{FF}_{_{inferred}}, σ_{FF}_{_{inferred}}²)

FC_{chrX}˜ [symbol] (μ_{FC}_{_{chrX}}, σ_{FC}_{_{chrX}}²)

FC_{chrY}˜ [symbol] (μ_{FC}_{_{chrY}}, σ_{FC}_{_{chrY}}²)

in which there is a systematic, depth dependent bias for fetal fraction, FF_{inferred}, predictions.

$${μ}_{{FF}_{inferred}} = {FF}_{t} - \frac{{α}_{{FF}_{inferred}}}{{β}_{{FF}_{inferred}}^{d}}$$

Where α_{FFi} and β_{FFi} are fit by downsampling data. Depth scaling corrections to the variances in the Gaussian probabilities is performed by calculating variances as follows where d is the total number of sequencing reads:

σ_{FC}_{_{chrX}}²=σ_{S}_{_{chrX}}²+FC_{chrX}σ_{d}_{_{chrX}}²/d+FC_{chrX}²σ_{f}_{_{chrX}}²

σ_{FC}_{_{chrY}}²=σ_{S}_{_{chrY}}²+FC_{chrY}σ_{d}_{_{chrY}}²/d+FC_{chrY}²σ_{f}_{_{chrY}}²

σ_{FF}_{_{inferred}}²=σ_{S}_{_{FFi}}²+σ_{d}_{_{FFi}}²/d

Fold changes and fetal fractions are converted according to a sex call,

$$( {μ}_{{FC}_{chrX}} , {μ}_{{FC}_{chrY}} ) = \{ \begin{matrix}( 1 - {FF}_{chrX} / 2 , {FF}_{chrY} / 2 ) & if sex call ∈ ( XY , XYY , XXY ) \\ ( 1 , 0 ) & otherwise\end{matrix}$$

$${FF}_{chrX} = ( 2 \cdot {FF}_{t} - {α}_{XY} ) / ( {R}_{XY} {β}_{XY} + 1 ) \\ {FF}_{chrY} = ( 2 \cdot {R}_{XY} {β}_{XY} {FF}_{t} + {α}_{XY} ) / ( {R}_{XY} {β}_{XY} + 1 )$$

where R_{XY}=CN_{chrY}/(2−CN_{chrX}). Where CN is the copy number of placental cells. The relationship between FF_{chrX} and FF_{chrY} can be assumed to not be one-to-one. The parameters are given flat, uniform priors. In one embodiment, depth scaling is of an expected variance for use in a Bayesian graphical model, and the depth can e the total sequencing read count.

w=(w_{XY}, w_{XX}, w_{XXY}, w_{XYY}, w_{X}, w_{XXX})

α_{FF}, β_{FF}˜Unif

σ_{S}_{_{FFi}}, σ_{d}_{_{FFi}}˜Unif

σ_{S}_{_{chrX}}², σ_{d}_{_{chrX}}², σ_{f}_{_{chrX}}²˜Unif

σ_{S}_{_{chrY}}², σ_{d}_{_{chrY}}², σ_{f}_{_{chrY}}²˜Unif

α_{XY}, β_{XY}˜Unif

Since the different sex classes exhibit unique signatures in allosomes (FF_chrX and FF_chrY), these signatures can be used this to make a sex prediction. Table 2 shows six canonical sex classes and the expected values for FF_chrX and FF_chrY for each class.

TABLE 2
Expectation of Fetal Fractions for Different Sex Hypotheses

| | Expected | Expected | Expected |
|---|---|---|---|
| Phenotype | FF_chrX | FF_chrY | FF_inferred |
| XX | 0 | 0 | FFₜᵣᵤₑ |
| XY | −FFₜᵣᵤₑ | +FFₜᵣᵤₑ | FFₜᵣᵤₑ |
| X | −FFₜᵣᵤₑ | 0 | FFₜᵣᵤₑ |
| XXX | +FFₜᵣᵤₑ | 0 | FFₜᵣᵤₑ |
| XXY | 0 | +FFₜᵣᵤₑ | FFₜᵣᵤₑ |
| XYY | −FFₜᵣᵤₑ | +2 × FFₜᵣᵤₑ | FFₜᵣᵤₑ |

The true fetal fraction for the sample is assumed to be FF_true.

The prior prevalence of the sex classes can be combined with the likelihood of the data for a given sex-calling hypothesis and constructed a posterior probability of a sex call (see Equation 1). In doing so, a generative model of fetal fraction measurements can be constructed from a true sex call according to a true fetal fraction in which a latent true fetal fraction (FFₜ) is postulated under which each FF measurement is conditionally independent from the other. And using the Bayesian theorem, the posterior probability of sex calls given the data for each sample can be computed.

P(SCA|FF_{chrX}, FF_{chrY}, FF_{inferred}, depth)∝P(SCA)P(FF_{chrX}, FF_{chrY}, FF_{inferred}, depth|SCAⱼ) (1)

Since the Bayesian sex caller (BSC) uses FF_{inferred} in this example implementation of a model, it can be capable of making sex hypotheses for vanishing twins (XXVT) or maternal mosaic monosomy X (X_MOS) (see Table 3). Vanishing twin syndrome occurs when a twin or multiple disappears in the uterus during pregnancy as a result of a miscarriage of one twin or multiple. The fetal tissue is absorbed by the other twin, multiple, placenta or the mother. This gives the appearance of a “vanishing twin.” Maternal mosaicism is the case that a subset of the mother's own cells have a deletion of a portion or all of chromosome X.

TABLE 3
Expectation of Fetal Fractions for Complex Sex Phenotypes

| | Expected | Expected | Expected |
|---|---|---|---|
| Phenotype | FF_chrX | FF_chrY | FF_inferred |
| XXVT | 0 | ν | FFₜᵣᵤₑ |
| X_MOS | −m × FFₜᵣᵤₑ | 0 | FFₜᵣᵤₑ |

ν is a constant for the expected value of FF_chrY for vanishing twins. m is a constant for the degree of mosaicism in X_MOS.

XXVT and X_MOS can be converted to report out as XX since that is the true sex chromosome status of the fetus in these particular scenarios.

For twins' sex calling, the pregnancy can be assumed to be a twin pregnancy and a sex prediction made according to the likelihood specified in Table 4. XX|XX means both twins are female, XX|XY means one fetus is male and the other female, and XY|XY means both twins are male.

TABLE 4
Expectation of Fetal Fraction for Twins

| Phenotype | FF_avg | FF_inferred | Note |
|---|---|---|---|
| XX\|XX | 0 | FFₜᵣᵤₑ | Twins, two XX |
| XX\|XY | ½ × FFₜᵣᵤₑ | FFₜᵣᵤₑ | Twins, one XX and one XY |
| XY\|XY | FFₜᵣᵤₑ | FFₜᵣᵤₑ | Twins, two XY |

In summary, the four variables can be used for each sample to make a sex prediction as described herein.
fold_change_chrX (equivalent of FF_chrX)
fold_change_chrY (equivalent of FF_chrY)
FF_inferred
total_mapped_reads

A model can consume these data and provide a set of posterior probabilities. The model then chooses the sex class for the highest posterior probability for each singleton and twin prediction. An example outcome for a sample is shown in Table 5. The singleton or twin status is provided at the time of ordering, and thus the appropriate sex prediction is reported.

TABLE 5
Output of a Bayesian Sex Call.

| Input | Value |
|---|---|
| fold_change_chrX | 0.95 |
| fold_change_chrY | 0.035 |
| FF_inferred | 0.1 |
| total_mapped_reads | 19000000 |

| Singleton Hypothesis | Output |
|---|---|
| FF_CALL_BAYES | XY |
| p_X | 2.920086905323199e−126 |
| p_XX | 6.282777958141852e−154 |
| p_XXVT | 1.092937510859974e−65 |
| p_XXX | 5.657385128531346e−180 |
| p_XXY | 5.717448576596899e−30 |
| p_XY | 0.995637316601831 |
| P_XYY | 1.710251562536671e−21 |
| p_X_MOS | 3.0923057329382e−138 |
| p_no_sex_call | 0.00436268339816771 |

| Twin Hypothesis | Output |
|---|---|
| TWIN_FF_CALL_BAYES | no_sex_call |
| twin_p_XX | 7.308659541856476e−158 |
| twin_p_XX_XY | 7.400987710250555e−07 |
| twin_p_XY | 0.0001158209668189907 |
| twin_p_no_sex_call | 0.999883438934411 |

If we assume a singleton pregnancy, the sex prediction for this particular sample with the FF measurements and depth is “XY.” If we assume a twin pregnancy, then no sex call is declared. FF_CALL_BAYES is a sex prediction for a singleton; TWIN_FF_CALL_BAYES is a twin sex prediction; p_<phenotype> is a posterior probability for the <phenotype>.

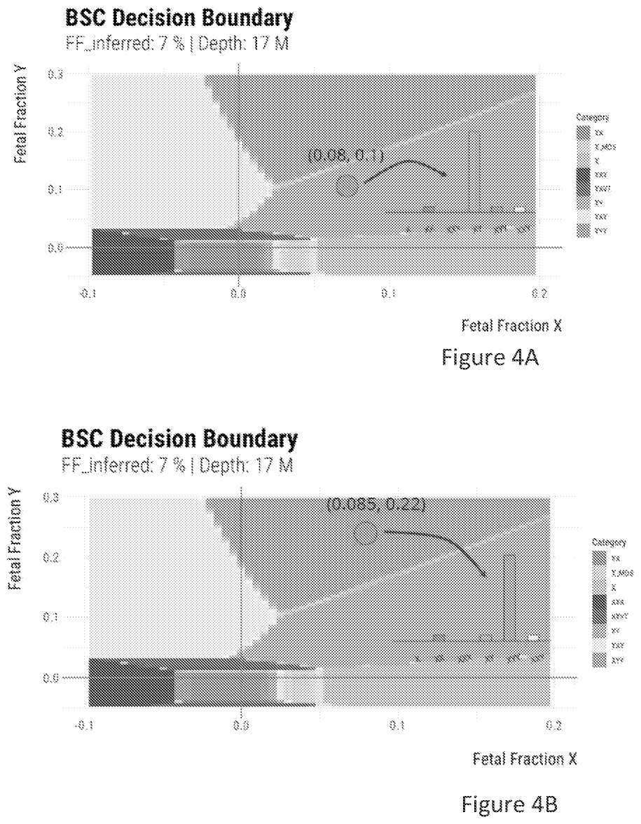

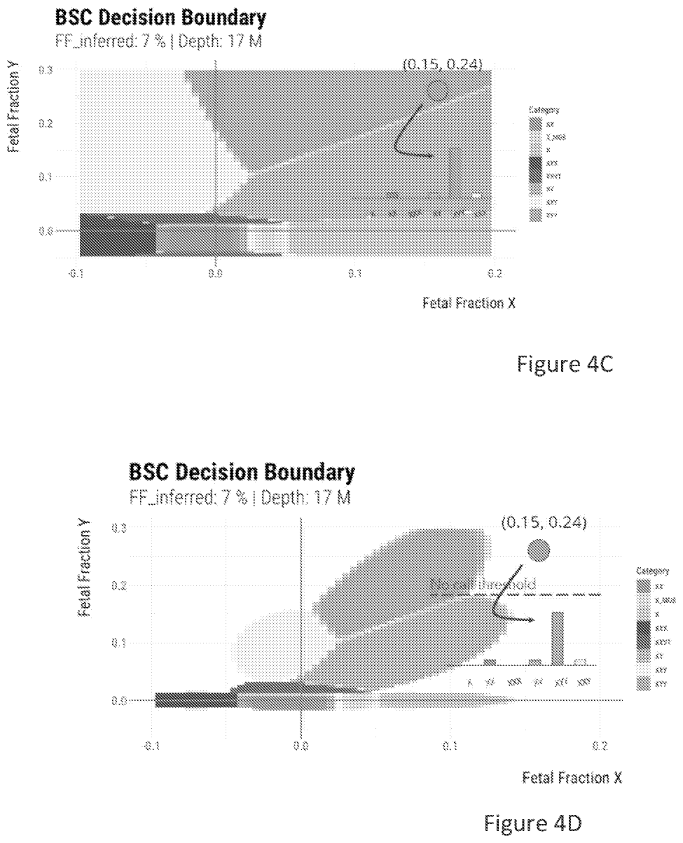

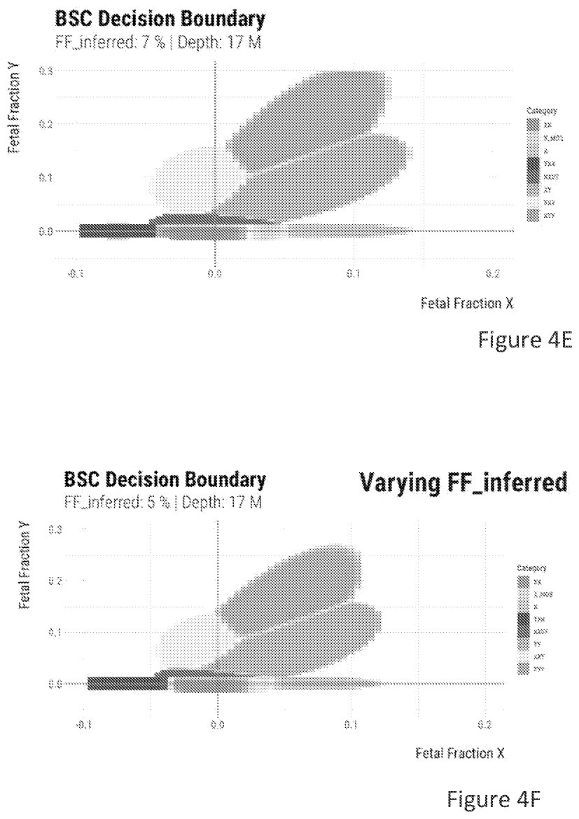

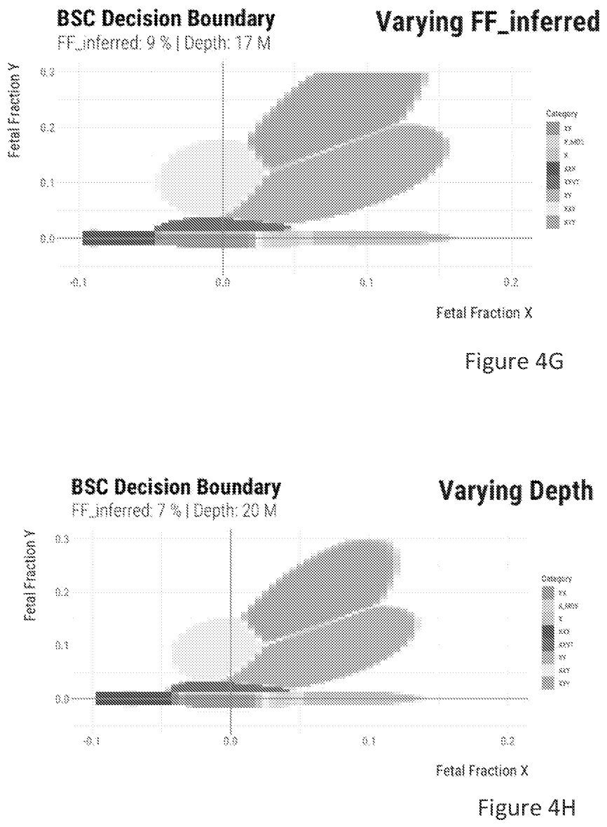

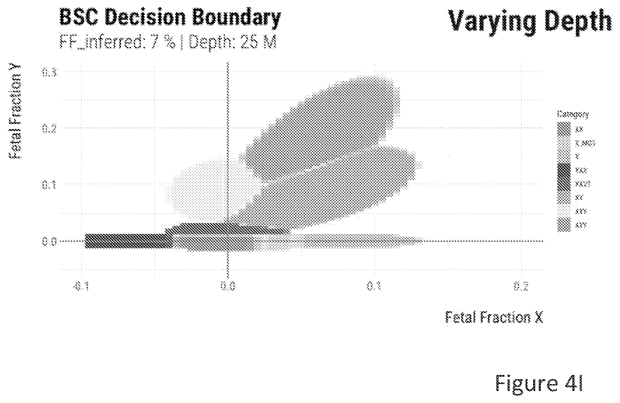

FIGS. 4A-4I are diagrams for visualization graphically showing results from patient samples. The axes on the graph include Fetal Fraction X along an x-axis and Fetal Fraction Y along a y-axis. A category of possible results is shown as a key and corresponds to similarly colored regions of the graph. The category key in this example includes results indicating XX shown in red, X_MOS shown in pink, X shown in orange, XXX shown in brown, XXVT shown in purple, CY shown in green, XXY shown in yellow, and XYY shown in blue. The color-coded key corresponds to similar colored regions of the graph as shown in FIGS. 4A-4I. A bar graph is also shown including relative probabilities for the various categories.

In FIG. 4A, for example, a patient sample is graphed at (0.08, 0.1) (Fetal Fraction X, Fetal Fraction Y). In this example, the patient sample is graphed in the green region corresponding to an XY call. The bar graph on the right shows the results from the Bayesian network showing the results indicating that the most likely category based on relative bar sizes. In this example the green bar is significantly larger than the other possible categories and the resulting call would correspond to the green key, i.e., an XY call.

FIG. 4B shows another patient sample graphed at (0.085, 0.22). In this example, the patient sample is graphed in the blue region corresponding to an XYY call. The embedded bar graph shows the results of the Bayesian network showing the results indicating the most likely category based on relative bar sizes. In this example, the blue bar is significantly larger than the other possible categories and the resulting call would correspond to the blue key, i.e. , an XYY call.

FIG. 4C shows anther patient sample graphed at (0.15, 0.24) near the boundary of the blue and green regions. The embedded bar graph shows a predominant blue bar, but compared to the corresponding bar shown in FIG. 4B is relatively lower indicating a less confident call. In this particular example, the resulting call would still correspond to the blue key, i.e., and XYY call but at a lower confidence level.

FIG. 4D shows yet another patient sample graphed at (0.15, 0.24). In this example, the graphed point for the patient results is outside the colored regions corresponding to the key. The embedded bar graph shows a threshold line, and none of the bars reach that threshold line. As a result the network makes a NO CALL indicating that no result was determined within a predetermined confidence level. Such samples are typically retested in a production workflow to resolve.

FIGS. 4A through 4D each correspond to a FF_{inferred} of 7% and a Depth of 17 million reads.

FIGS. 4E through 4G show decision boundary changes as a result of changes in Fetal Fraction Inferred. Specifically, FIG. 4E shows a set of decision boundaries for a FF_{inferred} of 7%, FIG. 4F shows another set of decision boundaries for a FF_{inferred} of 5%, and FIG. 4G shows yet another set of decision boundaries for a FF_{inferred} of 9%.

FIGS. 4H through 4I show decision boundary changes as a result of changes in depth. Specifically, FIG. 4H shows a set of decision boundaries for a depth of 20 M, and FIG. 4I shows a set of decision boundaries for a depth of 25 M with a common FF_{inferred} of 7%.

### EXAMPLE

SCA sensitivity, SCA specificity, and sex-calling accuracy were evaluated for singletons by using the clinical outcome data. For twins, the sex-calling accuracy was evaluated by using clinical outcome data on twins. Table 6 shows the number of SCAs in the pre-processed clinical outcome data that have been used in the validation.

TABLE 6
Number of SCAs in Clinical Singleton Outcomes Data

| Clinical SCA | Count | Percentage |
|---|---|---|
| X | 11 | 0.391% |
| XX | 1,383 | 49.1% |
| XXX | 4 | 0.142% |
| XXY | 7 | 0.249% |
| XY | 1,405 | 49.9% |
| XYY | 4 | 0.142% |

In this example, 57 twin samples met all the criteria. Table 7 shows the distribution of twin types (XX and XX pregnancy, one XX and one XY pregnancy, or XY and XY pregnancy) samples in the dataset.

TABLE 7
Number of Fetal Sex Calls in Clinical Twins Outcome Data

| Twin Types | Count | Percentage |
|---|---|---|
| XX XX | 15 | 26.3% |
| XX XY | 29 | 50.9% |
| XY XY | 13 | 22.8% |

### Table 7. Number of Fetal Sex Calls in Clinical Twins Outcome Data

The singleton data and the twin data were analyzed and compared them to known sex aneuploidy and sex calls. Each of the calls was labeled according to Table 2 and generate the relative metrics specified in Equation 2, Equation 3, Equation 4, and Equation 5.

$$Sensitivity of SCA i = \frac{{TP}_{i}}{{TP}_{i} + {\sum }_{i} {PN}_{i}} \qquad ( 2 )$$

$$Specificity of SCA i = \frac{\sum TN}{\sum TN + {\sum }_{i} {FP}_{i}} \qquad ( 3 )$$

$$SCA Call Accuracy = \frac{\sum {TP}_{i}}{\sum {TP}_{i} + {\sum }_{i} {WP}_{i}} \qquad ( 4 )$$

$$Sex - Call Accuracy = \frac{\sum TN}{\sum TN + \sum WS} \qquad ( 5 )$$

FIG. 3 illustrates an exemplary computing system or electronic device for implementing the examples of the disclosure. System 600 may include, but is not limited to known components such as central processing unit (CPU) 601, storage 602, memory 603, network adapter 604, power supply 605, input/output (I/O) controllers 606, electrical bus 607, one or more displays 608, one or more user input devices 609, and other external devices 610. It will be understood by those skilled in the art that system 600 may contain other well-known components which may be added, for example, via expansion slots 612, or by any other method known to those skilled in the art. Such components may include, but are not limited, to hardware redundancy components (e.g., dual power supplies or data backup units), cooling components (e.g., fans or water-based cooling systems), additional memory and processing hardware, and the like.

System 600 may be, for example, in the form of a client-server computer capable of connecting to and/or facilitating the operation of a plurality of workstations or similar computer systems over a network. In another embodiment, system 600 may connect to one or more workstations over an intranet or internet network, and thus facilitate communication with a larger number of workstations or similar computer systems. Even further, system 600 may include, for example, a main workstation or main general-purpose computer to permit a user to interact directly with a central server. Alternatively, the user may interact with system 600 via one or more remote or local workstations 613. As will be appreciated by one of ordinary skill in the art, there may be any practical number of remote workstations for communicating with system 600.

CPU 601 may include one or more processors, for example Intel® Core™ G7 processors, AMD FX™ Series processors, or other processors as will be understood by those skilled in the art (e.g., including graphical processing unit (GPU)-style specialized computing hardware used for, among other things, machine learning applications, such as training and/or running the machine learning algorithms of the disclosure; such GPUs may include, e.g., NVIDIA Tesla™ K80 processors). CPU 601 may further communicate with an operating system, such as Windows NT® operating system by Microsoft Corporation, Linux operating system, or a Unix-like operating system. However, one of ordinary skill in the art will appreciate that similar operating systems may also be utilized. Storage 602 (e.g., non-transitory computer readable medium) may include one or more types of storage, as is known to one of ordinary skill in the art, such as a hard disk drive (HDD), solid state drive (SSD), hybrid drives, and the like. In one example, storage 602 is utilized to persistently retain data for long-term storage. Memory 603 (e.g., non-transitory computer readable medium) may include one or more types of memory as is known to one of ordinary skill in the art, such as random access memory (RAM), read-only memory (ROM), hard disk or tape, optical memory, or removable hard disk drive. Memory 603 may be utilized for short-term memory access, such as, for example, loading software applications or handling temporary system processes.

As will be appreciated by one of ordinary skill in the art, storage 602 and/or memory 603 may store one or more computer software programs. Such computer software programs may include logic, code, and/or other instructions to enable processor 601 to perform the tasks, operations, and other functions as described herein (e.g., the monte carlo sampling of a posterior distribution from a Bayesian graphical model described herein), and additional tasks and functions as would be appreciated by one of ordinary skill in the art. Operating system 602 may further function in cooperation with firmware, as is well known in the art, to enable processor 601 to coordinate and execute various functions and computer software programs as described herein. Such firmware may reside within storage 602 and/or memory 603.

Moreover, I/O controllers 606 may include one or more devices for receiving, transmitting, processing, and/or interpreting information from an external source, as is known by one of ordinary skill in the art. In one embodiment, I/O controllers 606 may include functionality to facilitate connection to one or more user devices 609, such as one or more keyboards, mice, microphones, trackpads, touchpads, or the like. For example, I/O controllers 606 may include a serial bus controller, universal serial bus (USB) controller, FireWire controller, and the like, for connection to any appropriate user device. I/O controllers 606 may also permit communication with one or more wireless devices via technology such as, for example, near-field communication (NFC) or Bluetooth™. In one embodiment, I/O controllers 606 may include circuitry or other functionality for connection to other external devices 610 such as modem cards, network interface cards, sound cards, printing devices, external display devices, or the like. Furthermore, I/O controllers 606 may include controllers for a variety of display devices 608 known to those of ordinary skill in the art. Such display devices may convey information visually to a user or users in the form of pixels, and such pixels may be logically arranged on a display device in order to permit a user to perceive information rendered on the display device. Such display devices may be in the form of a touch screen device, traditional non-touch screen display device, or any other form of display device as will be appreciated be one of ordinary skill in the art.

Furthermore, CPU 601 may further communicate with I/O controllers 606 for rendering a graphical user interface (GUI) on, for example, one or more display devices 608. In one example, CPU 601 may access storage 602 and/or memory 603 to execute one or more software programs and/or components to allow a user to interact with the system as described herein. In one embodiment, a GUI as described herein includes one or more icons or other graphical elements with which a user may interact and perform various functions. For example, GUI 607 may be displayed on a touch screen display device 608, whereby the user interacts with the GUI via the touch screen by physically contacting the screen with, for example, the user's fingers. As another example, GUI may be displayed on a traditional non-touch display, whereby the user interacts with the GUI via keyboard, mouse, and other conventional I/O components 609. GUI may reside in storage 602 and/or memory 603, at least in part as a set of software instructions, as will be appreciated by one of ordinary skill in the art. Moreover, the GUI is not limited to the methods of interaction as described above, as one of ordinary skill in the art may appreciate any variety of means for interacting with a GUI, such as voice-based or other disability-based methods of interaction with a computing system.

Moreover, network adapter 604 may permit device 600 to communicate with network 611. Network adapter 604 may be a network interface controller, such as a network adapter, network interface card, LAN adapter, or the like. As will be appreciated by one of ordinary skill in the art, network adapter 604 may permit communication with one or more networks 611, such as, for example, a local area network (LAN), metropolitan area network (MAN), wide area network (WAN), cloud network (IAN), or the Internet.

One or more workstations 613 may include, for example, known components such as a CPU, storage, memory, network adapter, power supply, I/O controllers, electrical bus, one or more displays, one or more user input devices, and other external devices. Such components may be the same, similar, or comparable to those described with respect to system 600 above. It will be understood by those skilled in the art that one or more workstations 613 may contain other well-known components, including but not limited to hardware redundancy components, cooling components, additional memory/processing hardware, and the like.

Although implementations have been described above with a certain degree of particularity, those skilled in the art could make numerous alterations to the disclosed embodiments without departing from the spirit or scope of this invention. All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. A method for analyzing sex-chromosome aneuploidies of an individual comprising:
training a neural network model based on predetermined information related to at least one sex chromosome;
determining the respective sex-chromosome status based on a normalized read depth for a gene in a genome of the individual using a machine learning algorithm,
wherein the machine learning algorithm is configured to receive, as inputs, the normalized read depth, and output the respective sex-chromosome status of the individual.

2. The method of claim 1 wherein the operation of determining the respective sex-chromosome status is based on the normalized read depth and at least one of fetal fraction data and fold change data.

3. The method of claim 1 wherein the method comprises providing a twin sex calling.

4. The method of claim 3 wherein the twin sex calling comprises calling sexes among the following three phenotypes: two XX twins, two XY twins, and one XX twin and one XY twin.

5. The method of claim 1 wherein the method comprises determining a complex sex phenotype.

6. The method of claim 5 wherein the complex sex phenotype comprises at least one of the group comprising: vanishing twins and mosaic monosomy.

7. The method of claim 1 wherein the method provides a negative result where the respective sex-chromosome status is determined to be anomalous.

8. The method of claim 1 wherein the method determines the respective sex-chromosome status via Bayesian statistics of the read depth and allosome data.

9. The method of claim 1 wherein the method determines the respective sex-chromosome status via graphing of the read depth and allosome data.

10. The method of claim 9 wherein the operation of graphing comprises graphing a sample as a point in a two-dimensional plane.

11. The method of claim 1 wherein the method determines the respective sex-chromosome status via visualization of the read depth and allosome data.

12. The method of claim 11 wherein the visualization comprises graphing a sample as a point in a two-dimensional plane.

13. The method of claim 1 wherein the method comprises determining a probability of the sex-chromosome status for each sample of a plurality of samples according to the following:
P(SCA|FF_{chrX}, FF_{chrY}, FF_{inferred}, depth)∝P(SCA)P(FF_{chrX}, FF_{chrY}, FF_{inferred}, depth|SCAⱼ) (1).

14. The method of claim 1 wherein the determination of sex-chromosome status comprises heuristic data analysis and expert human review as a truth set.

15. The method of claims 1 wherein the predetermined information comprises human adjudicated sex-chromosome status.

16. The method of claim 15 wherein the human adjudicate sex-chromosome status calls are performed when the method provides a negative result.

17. The method of claim 1 wherein the operation of training comprises optimizing the Bayesian network model.

18. The method of claim 17 wherein the operation of optimizing comprises adapting learning rates based on a first and second gradient momentum.

19. The method of claim 1 wherein the operation of training comprises automated retraining protocols.

20. The method of claim 19 wherein the automated retraining protocol is adapted to synchronize the operation of training over time.

21. The method of any of claims 19 and 20 wherein the automated retraining protocol is adapted to reduce drift and repetitively validate performance over time.

22. The method of claim 1 wherein a confidence level is determined for the respective sex-chromosome status.

23. A system adapted to analyze sex-chromosome aneuploidies of an individual comprising:
a neural network model trained based on predetermined information related to at least one sex chromosome; the neural network model adapted to determine a respective sex-chromosome status based on a normalized read depth for a gene in a genome of the individual using a machine learning algorithm,
wherein the machine learning algorithm is configured to receive, as inputs, the normalized read depth, and output the respective sex-chromosome status of the individual.

24. The system of claim 23 wherein the neural network is adapted to determine the respective sex-chromosome status is based on the normalized read depth and at least one of fetal fraction data and fold change data.

25. The system of claim 23 wherein the neural network is adapted to provide a twin sex call.

26. The system of claim 25 wherein the twin sex call comprises a call of sexes among the following three phenotypes: two XX twins, two XY twins, and one XX twin and one XY twin.

27. The system of claim 23 wherein the neural network is adapted to determine a complex sex phenotype.

28. The system of claim 27 wherein the complex sex phenotype comprises at least one of the group comprising: vanishing twins and mosaic monosomy.

29. The system of claim 23 wherein the neural network is adapted to provide a negative result where the respective sex-chromosome status is determined to be anomalous.

30. The system of claim 23 wherein the neural network is adapted to determine the respective sex-chromosome status via Bayesian statistics of the read depth and allosome data.

31. The system of claim 23 wherein the method determines the respective sex-chromosome status via graphing of the read depth and allosome data.

32. The system of claim 31 wherein the operation of graphing comprises graphing a sample as a point in a two-dimensional plane.

33. The system of claim 23 wherein the neural network is adapted to determine the respective sex-chromosome status via visualization of the read depth and allosome data.

34. The system of claim 33 wherein the visualization comprises graphing a sample as a point in a two-dimensional plane.

35. The system of claim 23 wherein the neural network is adapted to determine a probability of the sex-chromosome status for each sample of a plurality of samples according to the following:
P(SCA|FF_{chrX}, FF_{chrY}, FF_{inferred}, depth)∝P(SCA)P(FF_{chrX}, FF_{chrY}, FF_{inferred}, depth|SCAⱼ) (1)

36. The system of claim 23 wherein the determination of sex-chromosome status comprises heuristic data analysis and expert human review as a truth set.

37. The system of claims 23 wherein the predetermined information comprises human adjudicated sex-chromosome status.

38. The system of claim 37 wherein the human adjudicate sex-chromosome status calls are performed when the method provides a negative result.

39. The system of claim 23 wherein the neural network is adapted to train based on an optimization of the Bayesian network model.

40. The system of claim 39 wherein the neural network is adapted to optimize based on an adaptation of learning rates based on a first and second gradient momentum.

41. The system of claim 23 wherein the neural network is adapted to train based on automated retraining protocols.

42. The system of claim 41 wherein the automated retraining protocol is adapted to synchronize the operation of training over time.

43. The system of any of claims 41 and 42 wherein the automated retraining protocol is adapted to reduce drift and repetitively validate performance over time.

44. The system of claim 1 wherein a confidence level is determined for the respective sex-chromosome status.
